(19) United States
(12) Patent Application Publication
(10) Pub. No.: US 2016/0022491 A1
(43) Pub. Date: Jan. 28, 2016

(54) Interventional Treatments for VitreoRetinal-Interface Syndromes

(71) Applicant: Strathspey Crown Holdings, LLC, Newport Beach, CA (US)

(72) Inventors: David Haydn Mordaunt, Los Gatos, CA (US); Andrew Brian Merkur, Vancouver (CA); David Tat-Chi Lin, Vancouver (CA)

(21) Appl. No.: 14/339,785

(22) Filed: Jul. 24, 2014

(51) Int. Cl. A61F 9/008

(52) U.S. Cl. CPC A61F 9/00802; A61F 2009/00851; A61F 2009/00863; A61F 2009/00874; A61F 2009/00885

(57) ABSTRACT

A system and method are provided for treating VitreoRetinal-Interface Syndromes (VRS) by using a femtosecond laser system to relieve vitreoretinal adhesions in an eye. Operationally, fibers in the vitreous body are severed by the laser system to create Posterior Vitreous Detachments (PVD) that relieve the adhesions. In a first embodiment for the present invention, tissue material on selected planes within the vitreous body is photoaltered to sever the fibers. Sequentially, or alternatively, to the first embodiment, in another embodiment, fibers at or near the vitreoretinal interface of the eye are photoaltered for this same purpose.

### FIELD OF THE INVENTION

The present invention pertains generally to systems and methods for performing ophthalmic surgical procedures using laser devices. More particularly, the present invention pertains to systems and methods for treating vitreous/retinal adhesions in the vitreous cavity of an eye. The present invention is particularly, but not exclusively, useful as a system and method for severing fibers in the vitreous cavity to relieve tension (traction) forces on the retina, to thereby prevent retinal detachments.

### BACKGROUND OF THE INVENTION

The retina is a sensory membrane that lines the inner eye at the back of the eye. The retina includes several layers. One such layer includes millions of rods and cones. In their combination, the rods and cones function to convert light that is focused on the retina into signals which are then transmitted to the brain by way of the optic nerve. In terms of size, the retina covers about 65 percent of the interior surface of the eye and includes the macula near its center. A dimple called the fovea is formed in the macula which includes cones, but not rods. Functionally, the macula and fovea provide the ability for a person to see fine details. This is an important portion of a person's vision, and is often referred to as central vision.

Inside the eye, the vitreous humor is a clear, viscous, gel-like material that fills the void in the eye between the retina and the crystalline lens. Embryologically, the vitreous serves as a scaffold for ocular development. After the first few decades of life, however, the vitreous gel starts to degenerate. With this degeneration, changes to the gelatinous nature of the vitreous body occur. In particular, as a person ages, the vitreous body can decompose or liquefy, and fibers can develop in the vitreous body. In turn, this aging process can cause the vitreous humor to undergo anomalous or partial separation from the retinal surface. When this happens, fibers in the vitreous body which have become attached to the retina are able to pull on various retinal structures in tangential, as well as anterior-posterior, directions. Vitreous pockets (enclosures) can then develop and fiber elements in these pockets will consequently exert traction forces on areas of the retinal surface. Anomalous posterior vitreous separation with residual traction on the optic disc or macula, as well as resultant fluid currents from ocular saciacic movements, can lead to a group of disorders which are collectively termed VitreoRetinal-Interface Syndromes (VRS). These include but are not limited to: epiretinal membrane, lamellar macular hole, full thickness macular hole, vitreopapillary and vitreomacular traction syndromes, symptomatic vitreomacular adhesion, peripheral retinal tears, vitreous hemorrhage from shearing or avulsing of retinal blood vessels and retinal detachment.

In the context of the present invention, the membranes at the interface between the vitreous humor and the retina are of particular concern. Respectively, these membranes are the cortex of the vitreous (i.e. cortical vitreous) and the Internal Limiting Membrane (ILM). As an anatomical structure, the cortex of the vitreous surrounds the vitreous humor, and it has a thickness that is in the range of 20-50 microns. It functions as a so-called “sac” which borders and defines the body of the vitreous humor. The ILM, on the other hand, overlies the retina in juxtaposition with the cortex of the vitreous.

Anatomically, the ILM is a relatively thin layer of tissue with a thickness of slightly more than 10-20 microns and, importantly, it does not contribute to the optical functionality of the retina. Normally, at their interface, the cortex of the vitreous and the ILM do not exert friction or traction forces on each other. With this in mind, however, the concern for the present invention arises when the cortex of the vitreous and the ILM adhere (i.e. attach or stick) to each other.

From an optical perspective, image perception by an eye relies on light that enters through the pupil and crystalline lens. This light is focused by the crystalline lens, and passes through the vitreous humor to be incident on the retina of the eye. An important portion of this focused light is directed onto the macula and the retinal tissue immediately surrounding the macula. As a practical matter, this light contributes most to the imaging capability of the eye. It will pass through the vitreous humor and be confined within what is hereinafter defined as an optical channel.

For purposes of the present invention, the optical channel will be generally cylindrical-shaped. It will have a cross-section diameter of greater than about 5 mm, and it will extend from the posterior surface of the crystalline lens to the ILM of the retina. Safety margins can be included with the optical channel and appropriately established around the optical channel.

In light of the above, it is an object of the present invention to provide a system and method for severing vitreous fibers that are attached to the retinal surface, to thereby prevent or alleviate the traction forces that cause VitreoRetinal-Interface Syndromes (VRS). Another object of the present invention is to provide a system and method for using a pulsed femtosecond laser to sever fibers in the vitreous humor. Still another object of the present invention is to provide interventional treatments for VRS that are easy to use, are simple to implement and are comparatively cost effective.

### SUMMARY OF THE INVENTION

In general, the purpose of the present invention is to provide a method, a system, and a set of executable instructions stored on a computer medium which will effectively eliminate traction forces that may develop between the vitreous humor and the retina. These forces can result for any of several reasons and can cause a variety of conditions, collectively referred to as VitreoRetinal-Interface Syndromes (VRS). For example, a detached retina is a VRS. As implied above, VRS conditions typically result from traction forces that are generated at the interface between the vitreous humor and the retina.

As envisioned for the present invention, traction forces resulting from vitreoretinal adhesions can be eliminated in either of several ways. For one, local areas of adhesion at the interface between the cortical vitreous and the Internal Limiting Membrane (ILM) of the retina can be directly photoablated by Laser Induced Optical Breakdown (LIOB) to remove the adhesive tissues. For another, fibers that form in the vitreous humor, and that pull on the retina to cause or aggravate VRS, can be severed by creating LIOB cutting planes in the vitreous humor. Further, bubbles which are formed in the vitreous humor during LIOB in the above-mentioned methodologies will coalesce into larger bubbles with high surface tension. These larger bubbles can then be further manipulated to facilitate release of residual vitreoretinal adhesion sites to thereby improve the efficacies of these methodologies.

Structurally, a system for severing fibers in the vitreous humor by LIOB includes a laser unit and a control unit for moving the focal point of a laser beam within the gelatinous material. In this combination, an imaging unit is provided for creating an anatomical profile of the vitreous humor of the eye. In particular, this anatomical profile will show the relationship of the vitreous humor with both the crystalline lens and the retina of the eye. Also included here is a programming unit that uses parameters obtained from the anatomical profile to define a laser pathway through the vitreous humor for use during the LIOB that is to be performed. A computer, which is connected in combination with both the imaging unit and the programming unit, obtains information respectively from these units regarding the anatomical profile and the pathway. The computer then uses this information for collective use in creating a control input to the laser unit. The control input is then transmitted to the laser unit, which, in response, generates a laser beam and moves the focal point of the laser beam along the pathway to perform the intended LIOB.

In detail, for one embodiment of the present invention, fibers that extend into the vitreous humor can be severed to relieve tension forces on the retina to prevent or reverse VRS. For this aspect of the present invention, a method for severing fibers can begin by first defining an optical channel that is characterized by an identified axis extending through the gelatinous material. For this purpose, the identified axis can be a visual axis, an optical axis, a central axis, or some other axis well known in the pertinent art which is anatomically oriented on the eye. Based on the selected axis, the optical channel is established to extend through the vitreous humor. Further, the optical channel is substantially cylindrical, or cone-shaped, and it extends radially outward to a distance r from the axis. Typically, r will be greater than about 5 mm. Preferably, the optical channels will overlie the macula for vitreomacular disorder but other channels will be defined to overlie (i.e. cover) the macula of the retina of the eye. Channels, along different axes, may be necessary to treat peripheral diseases.

With the optical channel defined, the method for severing fibers can include the step of establishing a first plane (or a plurality of mutually parallel first planes) in the gelatinous material that is/are oriented substantially perpendicular to the axis. Also, the method includes the step of establishing a second plane (or a plurality of mutually parallel second planes) in the gelatinous material that is/are oriented substantially parallel to the axis. Typically, the first and second planes are formed in a sequence so gas bubbles which are induced by LIOB do not interfere with the laser pattern. Next, material in the first and second planes is selectively photoablated to sever fibers in the gelatinous material.

For another embodiment of the present invention, a localized area of vitreoretinal adhesion is identified in the back of the eye. Specifically, the area of adhesion will typically be at the interface between the vitreous humor and the ILM of the retina (i.e. the vitreoretinal interface). Based on the location of the adhesion, a Target Tissue Volume (TTV) is identified that includes both a portion of the cortex of the vitreous and a portion of the ILM that are juxtaposed with each other in the area of vitreoretinal adhesion. In detail, the TTV will have a posterior surface that is located in the tissue of the adhesion and is oriented substantially parallel to the vitreoretinal interface. Further, the posterior surface of the TTV is located within a predetermined distance from the vitreoretinal interface.

Preferably, the posterior surface of the TTV can be located anterior to the vitreoretinal interface. It can happen, however, that the posterior surface will need to be located within the ILM of the retina, posterior to the vitreoretinal interface. In this latter case, the posterior surface of the TTV will still be oriented substantially parallel to the vitreoretinal interface. Further, in order to avoid delicate cellular elements of the retina, it will be important that the layer of ILM which is included in the TTV be less than approximately ten microns thick. As envisioned for the present invention, the location and orientation of the anterior surface of the TTV is discretionary.

Once the Target Tissue Volume (TTV) has been defined, a laser pathway is appropriately defined through the TTV. Photoablation of target tissue along the pathway in this volume will then eliminate the vitreoretinal adhesion in the localized area. As implied above, any bubbles that result from this protocol can be subsequently manipulated to enhance the efficacy of the protocol.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of the operative components of the present invention;

FIG. 2 is a cross-section view of an eye showing fibers extending from the back of the eye into the vitreous humor;

FIG. 3 is a perspective view of a photoablation pattern for use with the system and methodology of the present invention;

FIG. 4 is a top plan view of the photoablation pattern of FIG. 3;

FIG. 5 is a perspective view of an alternate photoablation pattern for use with the system and methodology of the present invention;

FIG. 6 is a top plan view of the photoablation pattern of FIG. 5;

FIG. 7 is a cross-section view of an eye showing a vitreoretinal adhesion;

FIG. 8 is a plan view of a portion of the fundus of the eye enclosed within the line 8-8 in FIG. 7;

FIG. 9A is a cross-section view of the fundus of the eye shown in FIG. 7, as seen along the line 9-9 in FIG. 8, showing a Target Tissue Volume (TTV) with its posterior surface anterior to the vitreoretinal interface;

FIG. 9B is a cross-section view of the fundus of the eye shown in FIG. 7, as seen along the line 9-9 in FIG. 8, showing a Target Tissue Volume (TTV) with its posterior surface established within the Internal Limiting Membrane (ILM) of the retina, posterior to the vitreoretinal interface;

FIG. 9C is a cross-section view of the fundus of the eye shown in FIG. 9A or FIG. 9B after a Posterior Vitreous Detachment (PVD) has developed; and

FIG. 10 is an operational flow chart for use with the present invention.

### DESCRIPTION OF THE PREFERRED EMBODIMENTS

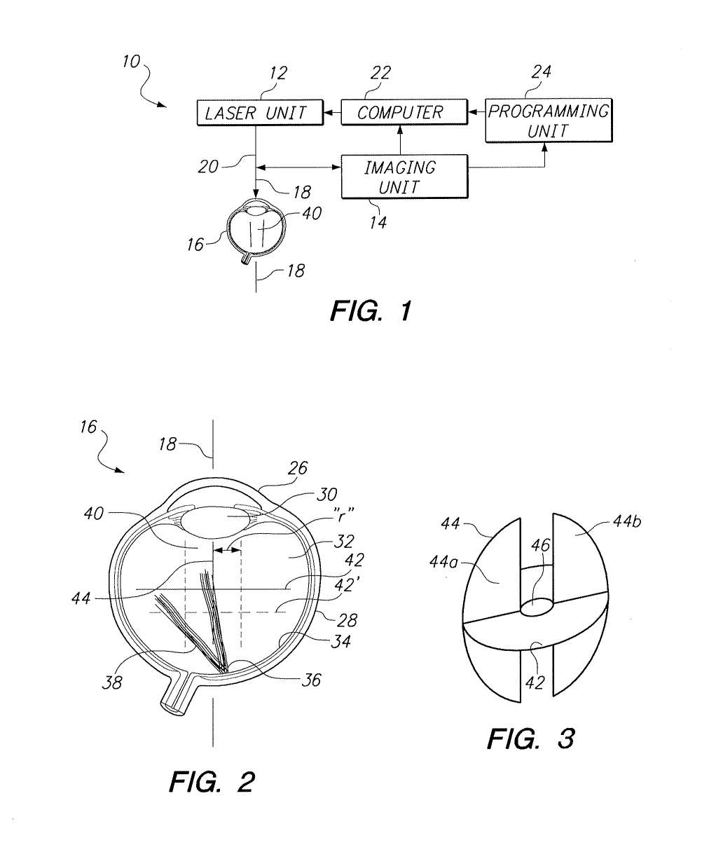

Referring initially to FIG. 1, a system in accordance with the present invention is shown and is generally designated 10. As shown, the system 10 includes a laser unit 12, and an imaging unit 14, that are each respectively positioned for optical interaction with an eye 16. More specifically, the laser unit 12 and the imaging unit 14 are positioned to direct their respective light beams along an axis 18.

For the present invention, the axis 18 is defined relative to selected anatomical features of the eye 16, and it will normally be a reference base that is well known in the pertinent art, such as a visual axis, a central axis or an optical axis. The laser unit 12 may also be of a type that is well known in the pertinent art and is capable of generating a pulsed femtosecond laser beam 20 (i.e. a beam having a sequence of laser pulses with ultra-short pulse durations [e.g. less than approximately 500 fs]). In particular, a laser beam 20 capable of passing through tissue to a subsurface focal point to perform Laser Induced Optical Breakdown (LIOB) of subsurface tissues in the eye 16 is to be used. In addition, the laser unit 12 can include a beam steering component for moving the focal spot of the laser beam 20 along a selected path to photoablate target tissue via LIOB. For example, the beam steering component can include a pair of mirrors (not shown) mounted on respective tip-tilt actuators to steer the laser beam 20 in respective, orthogonal directions. Further, the imaging unit 14 is typically of a type that is capable of creating a three-dimensional image of anatomical features in the eye 16, such as an Optical Coherence Tomography (OCT) imaging system, or any other suitable imaging device that is well known in the pertinent art such as a Scheimpflug device, a confocal imaging device, an optical range-finding device, an ultrasound device or a two-photon imaging device.

FIG. 1 also shows that the system 10 includes a computer 22 which is electronically connected with the imaging unit 14 and with the laser unit 12. A programming unit 24, which is electronically connected between the imaging unit 14 and the computer 22, is also included. In detail, the computer 22 receives input from both the imaging unit 14 and the programming unit 24, and it uses this input to control the laser unit 12 in accordance with a predetermined protocol. The programming unit 24 can include non-transitory, computer-readable medium (e.g. persistent memory) having executable instructions stored thereon that direct the computer 22 to perform the processes described herein.

Referring now to FIG. 2, several pertinent structures in the eye 16 are identified including the cornea 26, the sclera 28, the lens 30, the vitreous humor 32, the retina 34 and the macula 36. Together, the sclera 28 and retina 34 establish a container that holds the vitreous humor 32. FIG. 2 also shows that a plurality of fine fibers 38 extend from the macula 36 and into the vitreous humor 32. As explained above, these fibers 38 can create traction forces on the retina 34 that can cause the vitreous humor 32 to pull on the retina 34.

Continuing with reference to FIG. 2, an optical channel 40 is shown extending through the vitreous humor 32. As indicated above, the optical channel 40 is defined in its relationship with the axis 18. In detail, the optical channel 40 is substantially cylindrical shaped, and it is characterized by a variable radius r that extends radially outward from the axis 18. Typically, r will be greater than about 5 mm, and the optical channel 40 will be formed with a slightly increasing or decreasing taper as it extends in a posterior direction. With these dimensional characteristics, the optical channel 40 is established to extend through the vitreous humor 32. As shown, the optical channel 40 extends from the crystalline lens 30 of the eye 16 to the retina 34 of the eye 16 and covers (i.e. overlies), the macula 36 of the retina 34 with possible extension to the retinal periphery.

For an operation of the system 10 of the present invention, the imaging unit 14 is first used to create an anatomical profile of the vitreous humor 32 of the eye 16. Specifically, this anatomical profile identifies the dimensional relationship between the crystalline lens 30 and the retina 34 of the eye 16. The programming unit 24, which is electronically connected to the imaging unit 14, is used to locate the optical channel 40 in the vitreous humor 32. Once the optical channel 40 has been defined and located in the eye 16, the programming unit 24 defines pathway(s) (not shown) through the portion of the vitreous humor 32 that may be inside or outside the optical channel 40. Importantly, the pathway(s) is/are detailed according to parameters obtained from the anatomical profile that have been created by the imaging unit 14.

As noted above, the computer 22 is connected to the imaging unit 14, and to the programming unit 24. With these connections, the computer 22 obtains the necessary information regarding the anatomical profile and the pathway(s) that is/are required to create a control input for the laser unit 12. Operationally, this control input is then used by the laser unit 12 to generate the laser beam 20. The computer 22 also uses this control input for moving a focal point of the laser beam 20 along the pathway(s) in the vitreous humor 32. Specifically, all of this is done in accordance with the control input to operate the laser unit 12 for severing fibers 38 in the vitreous humor 32 without substantially disturbing the retina 34.

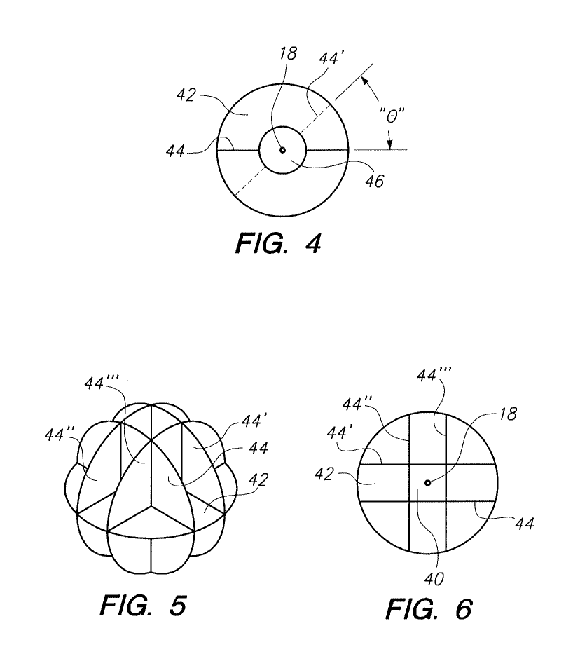

In more detail, as best appreciated by cross-referencing FIGS. 2 and 3, the method for severing fibers 38 can include the step of establishing one or more first planes 42, 42′ in the vitreous humor 32 that is/are oriented substantially perpendicular to the axis 18. Also, as shown, the method includes the step of establishing one or more second planes 44, 44′ (see FIG. 4 and description below) in the vitreous humor 32. As shown, the second planes 44, 44′ are either oriented substantially parallel to the axis 18, or they will intersect with the axis 18. In FIG. 3 it is shown that for an optional arrangement, the first plane 42 can be formed with a hole 46 to avoid intersection with the optical channel 40 (shown in FIGS. 1 and 2). For this arrangement the second plane 44 can include a pair of mutually coplanar sections 44a, 44b which are arranged to straddle the optical channel 40 (see FIG. 2). In this case the sections 44a and 44b are coplanar with the axis 18.

FIG. 4 shows an arrangement having a first plane 42 formed with a hole 46 and a pair of second planes 44, 44′ with second plane 44 positioned at a selected angle, θ, relative to axis 18, to second plane 44′. It will be appreciated that this arrangement of planes 44, 44′ will also pertain without the hole 46.

FIGS. 5 and 6 illustrate an arrangement in which fibers 38 (FIG. 2) are severed on a first plane 42 and four second planes 44, 44′, 44″, 44′″.

Once defined, material in the first plane(s) 42, 42′ (FIG. 2) and material in the second plane(s) 44, 44′, 44″, 44′″ (FIGS. 4 and 5) is selectively photoablated to sever fibers 38 in the vitreous humor 32. Specifically, this can be done by moving the focal point of a laser beam 20 (FIG. 1) along a pathway(s) within the first plane(s) 42, 42′ and second plane(s) 44, 44′, 44″, 44′″ to sever the fibers 38.

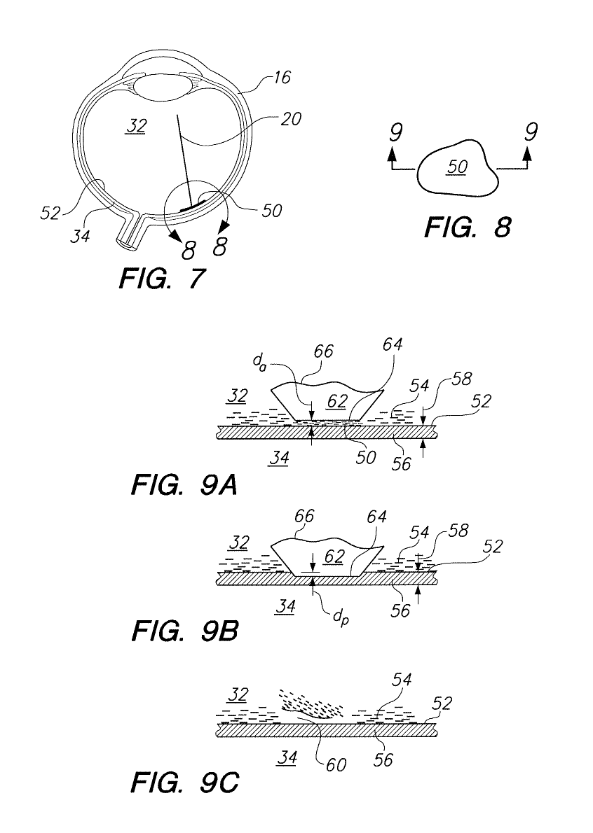

In another aspect of the present invention, it is understood that an adhesion 50 will sometimes form at the vitreoretinal interface 52 between the vitreous humor (vitreous body) 32 and the retina 34. Such an adhesion 50 may form for any of several reasons, and they are collectively referred to in the medical art as VitreoRetinal-Interface Syndromes (VRS). In the event, their common characteristic is that the adhesion 50 will create traction forces on the retina 34 that may eventually lead to damage or detachment of the retina 34. As indicated in FIG. 7, adhesions 50 occur in the back of the eye 16 and, as shown in FIG. 8, they can be extensive. In detail, the anatomical consequences of an adhesion 50 at the vitreoretinal interface 52 will perhaps be best appreciated with reference to FIG. 9A.

FIG. 9A shows that the vitreoretinal interface 52 is established by the cortex 54 of the vitreous body 32 (a.k.a. the cortical vitreous) and the Internal Limiting Membrane (ILM) 56 of the retina 34. Anatomically, the cortex 54 functions as a so-called “sac” for the vitreous body 32 and it varies in thickness through a range of about 20 μm to 50 μm. On the other hand, the thickness 58 of the ILM 56 is less than around 20 μm.

It is an important object for the present invention that tissue in the adhesion 50 of a VRS be photoablated for the purpose of separating the cortex 54 from the ILM 56. Specifically, this photoablation needs to be accomplished before traction forces in the adhesion 50 are able to somehow damage the retina 34. The intended result here is the creation of a Posterior Vitreous Detachment (PVD) 60 such as the one shown in FIG. 9C. In particular, the consequence of creating a PVD 60 is to sever fibers 38 that can form in the adhesion 50, and to thereby relieve traction forces on the retina 34 that could otherwise damage the retina 34.

Operationally, in accordance with the present invention, a PVD 60 can be initiated or developed by first defining a Target Tissue Volume (TTV) 62. Importantly, the TTV 62 will be defined with a posterior surface 64 that is located in the adhesion 50 and is oriented substantially parallel to the vitreoretinal interface 52. As envisioned for the present invention, the posterior surface 64 can extend completely across the extent of the adhesion 50 (see FIG. 8). On the other hand, the anterior surface 66 of the TTV 62 is somewhat indefinite and is essentially discretionary. FIGS. 9A and 9B indicate that, depending on the nature of the adhesion 50, and the depth to which fibers 38 have penetrated into the ILM 56 of the retina 34, the exact location of the posterior surface 64 of the TTV 62 may be varied. Specifically, in FIG. 9A, a situation is shown wherein the posterior surface 64 of the TTV 62 is established at a distance dₐ in the anterior direction from the vitreoretinal interface 52. On the other hand, in FIG. 9B, a situation is shown wherein the posterior surface 64 of the TTV 62 is established at a distance dₚ in the posterior direction from the vitreoretinal interface 52. In either case, the purpose is to photoablate tissue on the posterior surface 64 of the TTV 62 and thereby create a PVD 60 (see FIG. 9C), whereby the cortex 54 (vitreous body 32) is separated from the ILM 56 (retina 34) to prevent adverse traction forces from acting on the retina 34.

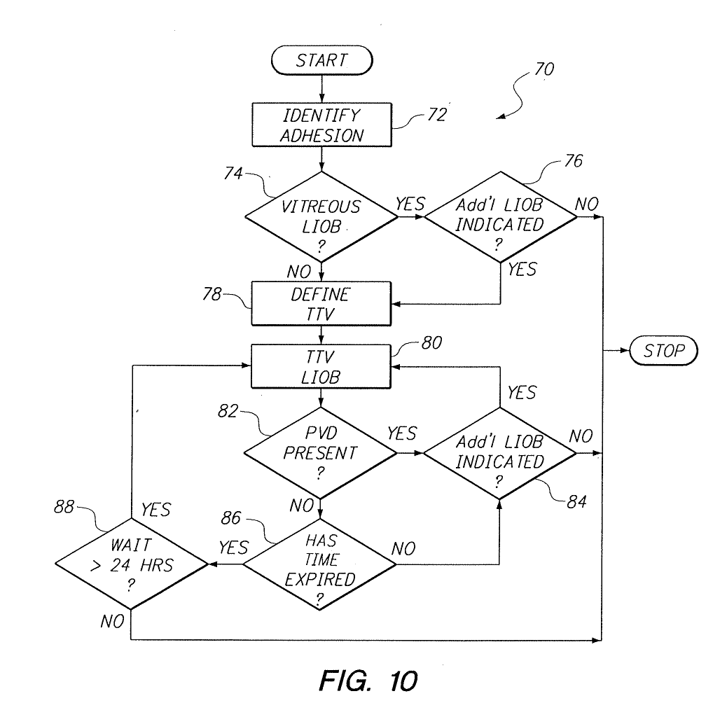

An operation of the present invention is perhaps best appreciated with reference to the operational flow chart which is shown in FIG. 10 and generally designated 70. In FIG. 10 it will be seen that after the start of a medical protocol (procedure) for the treatment of a VRS, block 72 of the chart 70 indicates that the first task to be accomplished is the identification of an adhesion 50. As envisioned for the present invention, the identification of an adhesion 50 will be accomplished essentially by the imaging unit 14. Once an adhesion 50 has been identified, inquiry block 74 then queries whether Laser Induced Optical Breakdown (LIOB) of the vitreous body 32 is required. If so, inquiry block 76 allows for the continued LIOB of tissue in the adhesion 50 to the extent necessary for a proper performance of tissue photoablation in the vitreous body 32.

In the event that LIOB in the vitreous body 32 is either not necessary (inquiry block 74), or requires augmentation (inquiry block 76), block 78 indicates a Target Tissue Volume (TTV) 62 needs to be defined. As envisioned for the present invention, the definition of the TTV 62 is essentially accomplished by the programming unit 24, using anatomical parameters pertinent to the vitreoretinal interface 52, the cortex 54 of the vitreous body 32, and the Internal Limiting Membrane (ILM) 56 of the retina 34, as disclosed above. Once the TTV 62 has been defined, block 80 indicates that LIOB is to be performed within the TTV 62.

As set forth in chart 70, and indicated by block 80, LIOB in the TTV 62 is performed for the specific purpose of creating a Posterior Vitreous Detachment (PVD) 60. Inquiry block 82 then indicates that the development of a PVD 60 is monitored. This monitoring may be done either visually, electronically (e.g. by using the imaging unit 14) or by a combination of both. If a PVD 60 has developed, the inquiry block 84 proceeds further to question whether continued LIOB in the TTV 62 is necessary. If not, the protocol is stopped. On the other hand, when no PVD 60 has yet developed, inquiry block 86 questions whether time has expired. This is a precautionary action that is taken to prevent, or limit, undue exposure of tissue to the photoablation effects of LIOB. When the procedure time has expired, inquiry block 88 indicates that the options are either to wait for at least an additional twenty-four hours before resuming the procedure, or to simply stop the procedure.

While the particular Interventional Treatments for VitreoRetinal-Interface Syndromes as herein shown and disclosed in detail are fully capable of obtaining the objects and providing the advantages herein before stated, it is to be understood that they are merely illustrative of the presently preferred embodiments of the invention and that no limitations are intended as to the details of construction or design herein shown other than as described in the appended claims.

What is claimed is:

1. A method for relieving traction forces caused by an adhesion between a container and fibers formed in a gelatinous material held within the container, the method comprising the steps of:
defining an axis extending through the gelatinous material;
establishing at least one plane extending through the gelatinous material inside the container, with the plane having a predetermined orientation relative to the axis; and
photoablating material in the plane to sever fibers in the gelatinous material for relieving the traction forces.

2. A method as recited in claim 2 wherein the establishing step further comprises the steps of:
establishing a first plane in the gelatinous material, wherein the first plane is oriented substantially perpendicular to the axis; and
establishing a second plane in the gelatinous material, wherein the second plane is oriented substantially parallel to the axis.

3. A method as recited in claim 2 further comprising:
a plurality of mutually parallel first planes; and
a plurality of second planes wherein each second plane in the plurality is parallel to at least one other second plane, and each second plane is at least a distance r from the axis.

4. A method as recited in claim 1 wherein the gelatinous material and the container are anatomical components of an eye, and wherein the gelatinous material forms the vitreous body of the eye.

5. A method for relieving an adhesion at the vitreoretinal interface in an eye of a patient, which comprises the steps of:
identifying and locating the adhesion;
defining a Target Tissue Volume (TTV) having a posterior surface and an anterior surface, wherein the posterior surface of the TTV is located in tissue of the adhesion and is substantially parallel to the vitreoretinal interface, and further wherein the posterior surface is located within a predetermined distance from the vitreoretinal interface;
photoaltering tissue in the TTV;
monitoring the TTV during the photoaltering step for a response signal indicative of the development of a Posterior Vitreous Detachment (PVD) at the posterior surface of the TTV; and
performing the photoaltering step in accordance with the response signal received during the monitoring step.

6. A method as recited in claim 5 wherein the posterior surface of the TTV is located anterior to the vitreoretinal interface and the predetermined distance is less than fifty microns.

7. A method as recited in claim 5 wherein the posterior surface of the TTV is located posterior to the vitreoretinal interface and the predetermined distance is less than ten microns.

8. A method as recited in claim 5 further comprising the step of stopping the photoaltering step when no response signal from the performing step has been received within a predetermined time duration established for the photoaltering step.

9. A method as recited in claim 8 further comprising the steps of:
waiting at least twenty four hours after the stopping step; and
restarting the method by resuming a sequential operation of the photoaltering step, the monitoring step, and the performing step.

10. A method as recited in claim 5 further comprising the step of paralyzing the eye during the photoaltering step.

11. A method as recited in claim 5 wherein the identifying step and the monitoring step are accomplished using Optical Coherence Tomography (OCT) techniques.

12. A method as recited in claim 5 wherein the photoaltering step further comprises the steps of:
generating a pulsed femtosecond laser beam having a wavelength and a predetermined power level for each pulse in the laser beam;
focusing the laser beam to a focal spot; and
using a pre-programmed computer for controlling the movement of the laser beam focal point along a predetermined path in the TTV, for the photoalteration of tissue in the TTV.

13. A system for severing fibers formed in the vitreous body of an eye to relieve traction forces on the retina in areas of vitreoretinal adhesions created by an interaction of the fibers with the retina, the system comprising:
an imaging unit for creating an anatomical profile of the vitreous body in its relationship with the crystalline lens and the retina of the eye, to include identifying an area of vitreoretinal adhesion and any fibers extending therefrom into the vitreous body, and further for defining an axis extending through the vitreous body;
a programming unit connected to the imaging unit for defining a pathway through the vitreous body relative to the axis;
a computer connected to the imaging unit, and to the programming unit, to obtain information therefrom regarding the anatomical profile, the location of the vitreoretinal adhesions and the pathway therethrough for severing fibers for creating a control input; and
a laser unit for generating a pulsed femtosecond laser beam, and for moving a focal point of the laser beam along the pathway within the vitreous body in accordance with the control input to selectively photoablate material and fibers to develop a Posterior Vitreous Detachment (PVD) in the area of vitreoretinal adhesion for relief of a VitreoRetinal-Interface Syndrome (VRS).

14. A system as recited in claim 13 wherein the control input causes the laser unit to selectively photoablate material on at least one plane extending through the gelatinous material inside the container, with the plane having a predetermined orientation relative to the axis.

15. A system as recited in claim 14 wherein photoablation is accomplished in a first plane oriented substantially perpendicular to the axis, and in a second plane oriented substantially parallel to the axis.

16. A system as recited in claim 13 wherein the control input causes the laser unit to photoablate material at the vitreoretinal interface of the eye within a Target Tissue Volume (TTV) having a posterior surface and an anterior surface, wherein the posterior surface of the TTV is located in tissue of the adhesion and is substantially parallel to the vitreoretinal interface, and further wherein the posterior surface is located within a predetermined distance from the vitreoretinal interface.

17. A system as recited in claim 13 wherein the imaging unit is a device selected from the group consisting of an Optical Coherence Tomography (OCT) device, a Scheimpflug device, a confocal imaging device, an optical range-finding device, an ultrasound device and a two-photon imaging device.

18. A non-transitory, computer-readable medium having executable instructions stored thereon that direct a computer system to perform a process comprising:
defining an axis extending through a gelatinous material held within a container, wherein the gelatinous material includes fibers interacting with the container to generate traction forces therebetween;
establishing at least one plane extending through the gelatinous material inside the container, with the plane having a predetermined orientation relative to the axis; and
photoablating material in the plane to sever fibers in the gelatinous material for relieving the traction forces.

19. A medium as recited in claim 18 and the process further comprises:
establishing a first plane in the gelatinous material, wherein the first plane is oriented substantially perpendicular to the axis;
establishing a second plane in the gelatinous material, wherein the second plane is oriented substantially parallel to the axis; and
photoablating material in the first and second planes.

20. A medium as recited in claim 18 wherein the container and the gelatinous material are components of an eye, and wherein the process further comprises photoablating material at the vitreoretinal interface of the eye within a Target Tissue Volume (TTV) having a posterior surface and an anterior surface, wherein the posterior surface of the TTV is located in the material and is oriented substantially parallel to the vitreoretinal interface, and further wherein the posterior surface is located within a predetermined distance from the vitreoretinal interface.
